(19) United States
(12) Patent Application Publication
(10) Pub. No.: US 2006/0153815 A1
(43) Pub. Date: Jul. 13, 2006

(54) Tissue engineering devices for the repair and regeneration of tissue

(76) Inventors: Agnieszka Seyda, New Brunswick, NJ (US); David C. Colter, Hamilton, NJ (US); Charito S. Buensuceso, North Brunswick, NJ (US); Sridevi Dhanaraj, Raritan, NJ (US); Anna Gosiewska, Skillman, NJ (US); Jeffrey C. Geesin, Doylestown, PA (US); Angelo Scopelianos, Whitehouse Station, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/304,091

(22) Filed: Dec. 15, 2005

Related U.S. Application Data
(60) Provisional application No. 60/637,781, filed on Dec. 21, 2004.

(51) Int. Cl. A61K 35/12; C12N 5/08; A61F 2/00

(52) U.S. Cl. 424/93.7; 424/423; 435/366; 435/368; 435/370; 435/372

(57) ABSTRACT

Tissue engineering devices for use in the repair or regeneration of tissue made of support scaffolds and cell sheets.

### FIELD OF THE INVENTION

The present invention relates to tissue engineering devices for use in the repair or regeneration of tissue. Specifically, this invention relates to tissue engineering devices made of support scaffolds and cell sheets.

### BACKGROUND OF THE INVENTION

Considerable attention and effort has recently been directed to the substitution of defective organs with operationally effective replacements. The replacements have ranged from completely synthetic devices such as artificial hearts to completely natural organs from another mammalian donor. The field of heart transplants has been especially successful with the use of both synthetic hearts to natural hearts from living donors. Equal success has not been achieved in many other organ fields.

The recent emergence of tissue engineering and regenerative medicine offers alternative approaches to repair and regenerate/restore damaged or diseased tissue form and function. Tissue engineering strategies have explored the use of biomaterials in combination with cells and/or growth factors to develop biological substitutes that ultimately can restore or improve tissue function. The use of colonizable and remodelable scaffolding materials has been studied extensively as tissue templates, conduits, barriers, and reservoirs. In particular, synthetic and natural materials in the form of foams, sponges, gels, hydrogels, textiles, and non-wovens have been used in vitro and in vivo to reconstruct/regenerate biological tissue, as well as deliver chemotactic agents for inducing tissue growth. In some cases, cells are loosely seeded onto the scaffolding materials and immediately implanted into the body. Alternatively, cells may be loosely seeded onto the scaffolding materials and cultured for a period of time prior to implantation.

Loosely seeding cells, however, lack the ability to control the distribution of cells on the surface of the scaffold, especially if a uniform distribution is required. What is needed is a device in which the distribution of cells on the surface of the scaffold is controllable. Furthermore, viability of loosely seeded cells might be compromised since cells require supportive factors such as extracellular matrix proteins for attachment, proliferation and migration.

### SUMMARY OF THE INVENTION

The present invention is a tissue engineering device for use in the repair or regeneration of tissue having a support scaffold layer and a cell sheet layer.

### BRIEF DESCRIPTION OF THE FIGURES

FIG. 1 is a schematic drawing of one embodiment of the present invention.

FIG. 2 is an outline of a method of making the tissue engineering device of the present invention.

### DETAILED DESCRIPTION OF THE INVENTION

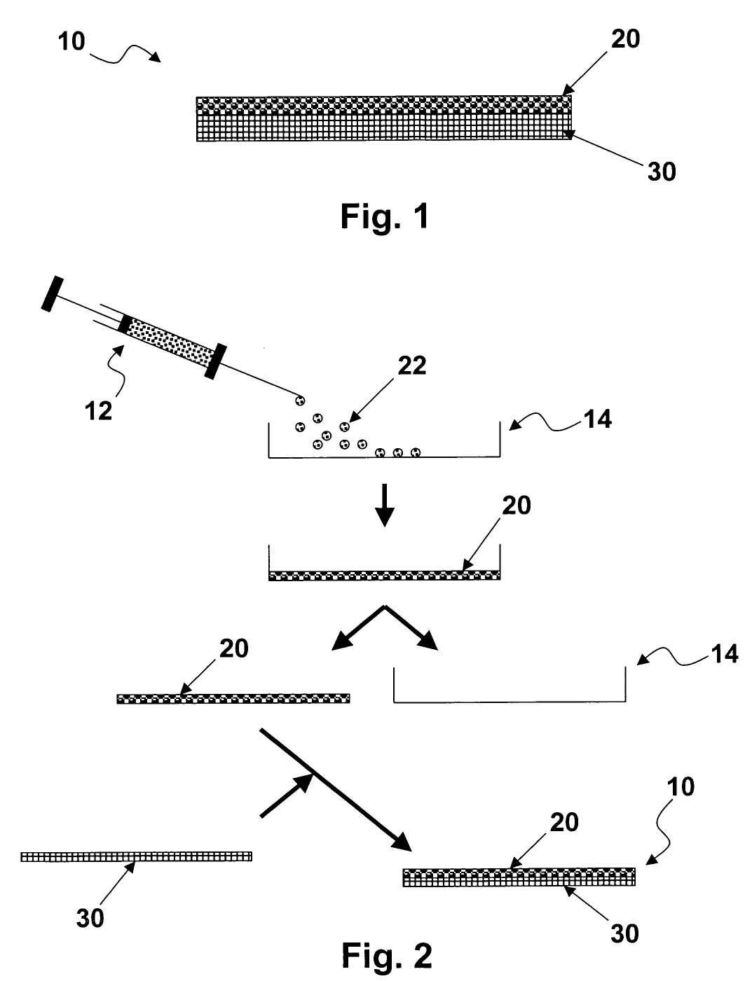

The present invention is a tissue engineering device for use in the repair or regeneration of tissue comprised of a support scaffold layer and a cell sheet layer. FIG. 1 is a schematic drawing of one embodiment of the present invention. The figure shows tissue engineering device 10 comprised of cell sheet layer 20 and scaffold layer 30.

A method of making tissue engineering device 10 of the present invention is shown in FIG. 2. Selected cells 22 are seeded on a cell growth support device 14, such as a Petri dish, by use of an inoculation device 12, typically a syringe or a pipette. Cells 22 are grown to over 85% confluence, preferably about 100% confluence, on cell growth support device 14. The over 85% confluent cells form cell sheet layer 20 on cell growth support device 14. Cell sheet layer 20 is then separated from cell growth support device 14, and disposed on scaffold layer 30 to create tissue engineering device 10.

Selected cells 22 may be autologous or may be derived allogeneically or xenogeneically.

Cells 22 may be obtained by treatment of an appropriate organ or tissue that is to serve as the source of the cells. Techniques for treatment of an organ or tissue to obtain stromal cells are known to those skilled in the art. For example, the tissue or organ can be mechanically disrupted and/or treated with digestive enzymes or chelating agents to weaken the interactions between cells making it possible to obtain a suspension of individual cells. Typically the method will include a combination of mechanical disruption, enzyme treatment and chelating agents. In one technique the tissue or organ is minced and treated simultaneously or subsequently with any of a number of digestive enzymes either alone or in combination. Examples of enzymes useful in dissociating cells include, but are not limited to, trypsin, chymotrypsin, collagenase, elastase, hyaluronidase, DNase, pronase, dispase, and the like. Mechanical disruption can also be accomplished by, for example, the use of blenders, sieves, homogenizers, pressure cells, and the like.

The resulting suspension of cells and cell clusters can be further divided into populations of substantially homogenous cell types. This can be accomplished using standard techniques for cell separation including, for example, positive selection methods (e.g., clonal expansion and selection of specific cell types), negative selection (e.g., lysis of unwanted cells), separation based upon specific gravity in a density solution, differential adherence properties of the cells in the mixed population, fluorescent activated cell sorting (FACS), and the like. Cells obtained by these methods are further expanded in culture using standard cell culture techniques known to those skilled in the art to obtain sufficient cell numbers as required for seeding on to the dishes.

Alternatively, cells may be obtained through a point of care approach.

Where autologous cells are used considerations of immunological response by the body are minimized or eliminated, while using allogeneic or xenogeneic cells may require further consideration of such immunological response by the body. Such a treatment strategy may require adjunct immunotherapy with cyclosporine, FK-506 or other such immunotherapeutic agents. Alternatively, cells might be transfected with genetic material, which is capable of reducing or eliminating an immune response in the host.

Selected cells 22 that may be utilized according to the present invention include, but are not limited to, chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, intervertebral disc cells, oral and gastrointestinal mucosal epithelia, urinary tract epithelia, as well as epithelia derived from other organ systems, skeletal joint synovium, periosteum, perichondrium, muscle cells (e.g. skeletal, smooth, cardiac), pericardium, dura, meninges, keratinocytes precursor cells, pericytes, glial cells, neural cells, amniotic and placental membranes, serosal cells (e.g., serosal cells lining body cavities), undifferentiated or pre-differentiated stem or progenitor cells, neural stem or progenitor cells, neuronal cells, dendritic cells, and genetically engineered cells. Stem cells include, without limitation, hematopoietic, mesenchymal, postpartum, pancreatic, hepatic, retinal epithelial, olfactory bulb, endothelial, muscle, adipose-derived, ileac crest, bone marrow, periodontal ligament, oval and dermal stem cells and organ specific stem cells or progenitor cells.

Cells of any animal, including arthropods (insects), can be used, as well as germs and embryos can also be used. Examples include, embryonic stem cells, somatic stem cell, neural stem cell, melanocytes, vascular smooth muscle cells, hair metrocytes, stellate cells, small hepatocytes, amnion-derived cells, fetal liver-derived cells, fetal kidney-derived cells, and fetal lung-derived cells, as well as, as cells of established cell lines, HeLa cells, FL cells, KB cells, HepG2 cells, WI-88 cells, MA104 cells, BSC-1 cells, Vero cells, CV-1 cells, BHK-21 cells, L cells, CHL cells, BAE cells, BRL cells, PAE cell and the like.

In one embodiment, a genetically engineered cell sheet layer 20 is prepared. The genetically engineered cell sheet layer 20 comprises a population of cells 22 wherein at least one cell of the population of cells is transfected with an exogenous polynucleotide such that the exogenous polynucleotide expresses express diagnostic and/or therapeutic product (e. g., a polypeptide or polynucleotide) to assist in tissue healing, replacement, maintenance and diagnosis. Examples of “proteins of interest” (and the genes encoding same) that may be employed herein include, without limitation, cytokines, growth factors, chemokines, chemotactic peptides, tissue inhibitors of metalloproteinases, hormones, angiogenesis modulators either stimulatory or inhibitory, immune modulatory proteins, neuroprotective and neuroregenerative proteins and apoptosis inhibitors. More specifically, preferred proteins include, without limitation, erythropoietin (EPO), EGF, VEGF, FGF, PDGF, IGF, KGF, IFN-α, IFN-δ, MSH, TGF-α, TGF-β, TNF-α, IL-1, BDNF, GDF-5, BMP-7 and IL-6.

In another aspect of the invention, cells 22 in cell sheet layer 20, and their progeny, can be engineered to continually produce a desired gene product. In another aspect the cells may only transiently produce a product. Methods of transforming or transfecting cells with exogenous polynucleotide such as a DNA molecule are well known in the art and include techniques such as calcium-phosphate- or DEAE-dextran-mediated transfection, protoplast fusion, electroporation, liposome mediated transfection, direct microinjection and adenovirus infection. The most widely used method is transfection mediated by either calcium phosphate or DEAE-dextran. Methods of permanent and transient transfections are known and will be described further herein. One particular advantage of utilizing a tissue sheet comprising genetically engineered cells is that a large amount of cells may be utilized compared to simply seeding a relatively few number of genetically engineered cells. In another preferred embodiment, a cell preparation graft would include a plurality of modified cell types, each producing a different therapeutic substance at a desired therapeutic level. This embodiment is useful as a screen for drug development.

In another aspect of the invention cell sheet layer 20 can be comprised of cells 22 reinforced with exogenously added extracellular matrix proteins, e.g., collagens, laminins, fibronectin, vitronectin, tenascin, integrins, glycosaminoglycans (hyaluronic acid, chondroitin sulfate, dermatan sulfate, heparan sulfate, heparin, keratan sulfate and the like), elastin and fibrin. In some embodiments of the invention cell sheet layer 20 can incorporate exogenously added growth factors and/or cytokines, such as vascular endothelial cell growth factors, platelet derived growth factors, epidermal growth factors, fibroblast growth factors, hepatocyte growth factors, insulin-like growth factors, and transforming growth factors.

Selected cells 22 may be cultured on a surface of glass, ceramic or a surface-treated synthetic polymer. For example, polystyrene that has been subjected to a surface treatment, like gamma-ray irradiation or silicon coating, may be used as a surface for cell culture. Cells 22 grown to over 85% confluence form cell sheet layer 20 on cell growth support device 14. Cell sheet layer 20 may be separated from cell growth support device 14 using proteolysis enzymes, such as trypsin or dispase. Non-enzymatic cell dissociation could also be used. A non-limiting example includes a mixture of chelators sold under the tradename CELLSTRIPPER (Mediatech, Inc., Herndon, Va.), a non-enzymatic cell dissociation solution designed to gently dislodge adherent cells in culture while reducing the risk of damage associated with enzymatic treatments.

Alternatively, the surface of cell growth support device 14, from which cultured cells 22 are collected, may be a bed made of a material from which cells detach without a proteolysis enzyme or chemical material. The bed material may comprise a support and a coating thereon, wherein the coating is formed from a polymer or copolymer which has a critical solution temperature to water within the range of 0° C. to 80° C.

The critical solution temperature is defined as follows. When a certain material is mixed with water, the mixture is divided into two layers at a s particular temperature because of its poor solubility, but eventually the material is completely dissolved with water to turn it to a uniform solution if it is either heated or cooled beyond a certain temperature. The certain temperature is defined as “critical solution temperature”. If the uniform solution is formed when heated, the critical solution temperature is called “upper critical solution temperature”. If the uniform solution is formed when cooled, it is called the “lower critical solution temperature”.

The polymer or copolymer should have either an upper or lower critical solution temperature within the range of 0° to 80° C., preferably 20° to 50° C. If it is higher than 80° C., cultured or grown cells may die. If it is lower than 0° C., the growth rate of the cells may be very much lowered or the cells may die.

The polymer or copolymer may be prepared by polymerizing or copolymerizing some hydrophilic monomers. Non-limiting examples of the monomers are represented by an acrylamide, such as methacrylamide; an N-substituted acrylamide or methacrylamide, such as N-ethyl acrylamide, N-n-propyl acrylamide, N-n-propyl methacrylamide, N-isopropyl acrylamide, N-isopropyl methacrylamide, N-cyclopropyl acrylamide, N-cyclopropyl methacrylamide, N-ethoxyethyl acrylamide, N-ethoxyethyl methacrylamide, N-tetrahydrofurfuryl acrylamide, N-tetrahydro furfuryl methacrylamide; an N,N-di-substituted acrylamide or methacrylamide, such as N,N-dimethyl acrylamide, N,N-dimethyl methacrylamide, N,N-ethylmethyl acrylamide, N,N-diethyl acrylamide; 1-(1-oxo-2-propenyl)-pyrrolidine, 1-(1-oxo-2-propenyl)-piperidine, 4-(1-oxo-2-propenyl)-morpholine, 1-(1-oxo-2-methyl-2-propenyl)-pyrrolidine, 1-(1-oxo-2-methyl-2-propenyl)-piperidine, 4-(1-oxo-2-methyl-2-propenyl)-morpholine etc.; a vinyl ether, such as methyl vinyl ether; and the like.

Additional monomers include acrylamide derivatives of the following general formula (I): 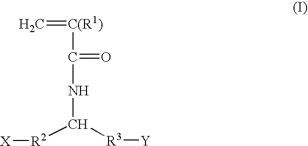
wherein R¹ represents a hydrogen atom, a straight-chain or branched alkyl group containing 1 to 6 carbon atoms or a C3-6 cycloalkyl group, R² and R³ each independently represent an alkylene group containing 1 to 6 carbon atoms or R² and R³ may be combined to form a ring, X represents a hydrogen atom, an amino group, a hydroxyl group, a halogen atom, a carboxyl group or a —COOR⁴ group wherein R⁴ represents a C1-6 straight-chain or branched alkyl, C3-6 cycloalkyl, phenyl, substituted phenyl, benzyl or substituted benzyl group, and Y represents an amino group, a hydroxyl group, a halogen atom, a carboxyl group or a —COOR⁴ group wherein R⁴ represents a C1-6 straight-chain or branched alkyl, C3-6 cycloalkyl, phenyl, substituted phenyl, benzyl or substituted benzyl group.

The polymer may also consist of identical or different repeating units of the following general formula (II): 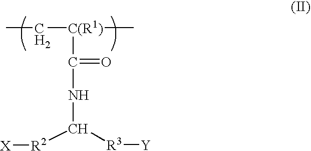
wherein R¹ represents a hydrogen atom, a straight-chain or branched alkyl group containing 1 to 6 carbon atoms or a C3-6 cycloalkyl group, R² and R³ each independently represent a alkylene group containing 1 to 6 carbon atoms or R² and R³ may be combined to form a ring, X represents a hydrogen atom, an amino group, a hydroxyl group, a halogen atom, a carboxyl group or a —COOR⁴ group wherein R⁴ represents a C1-6 straight-chain or branched alkyl, C3-6 cycloalkyl, phenyl, substituted phenyl, benzyl or substituted benzyl group, and Y represents an amino group, a hydroxyl group, a halogen atom, a carboxyl group or a —COOR⁴ group wherein R⁴ represents a C1-6 straight-chain or branched alkyl, C3-6 cycloalkyl, phenyl, substituted phenyl, benzyl or substituted benzyl group.

The copolymer may also consist of different or identical repeating units of the above general formula (II) and different or identical repeating units of general formula (III): 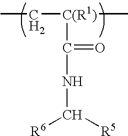
wherein R¹ represents a hydrogen atom, a straight-chain or branched alkyl group containing 1 to 6 carbon atoms or a C3-6 cycloalkyl group, R⁵ represents a straight-chain or branched alkyl group containing 1 to 6 carbon atoms or a C3-6 cycloalkyl group, and R⁶ represents a straight-chain or branched alkyl group containing 1 to 6 carbon atoms or a C3-6 cycloalkyl group, or R⁵ and R⁶ may be combined to form a 3-, 4-, 5 or 6-membered ring in which the —CH— group to which they are attached is one member.

A copolymer of the above listed monomers or other monomers, a graft polymer or copolymer or a mixture of the polymers can also be employed in order to adjust the critical solution temperature, to enhance an interaction between the support and the coating thereon or to control the balance between the hydrophilic and hydrophobic properties of the bed material. The polymer or copolymer of the present invention may be crosslinked unless the inherent properties of the polymer would be deleteriously affected thereby.

The support can be prepared from any material, for example polymers such as polystyrene, poly(methyl methacrylate), polyvinylidene difluoride (PVDF), polypropylene, polyethylene, vinyl polymers; ceramics, metals, glass and modified glass. The shape of cell growth support device 14 is not limited, but typically a Petri dish, a plate, a fiber, particles, and any type container can be used for the cell culture (e.g. a flask).

A polymer or copolymer can be bound on the support by a chemical method or by a physical method. In the chemical method an electron beam, gamma-ray irradiation, ultraviolet irradiation, corona treatment and plasma treatment can be used. In case where the support and the coating have groups reactive with each other, an organic reaction (e.g. a radical, anionic or cationic reaction) can also be used. In the physical method, the polymer per se or a combination of the polymer and a matrix compatible with the support is coated on the support, thus binding by physical absorption power. Examples of the matrix are graft or a block copolymer of the polymer to be coated, with the monomer forming the support or other monomers compatible with the support.

In order to collect or detach the grown or cultured cell sheet layer 20, the bed material is either heated or cooled to exceed the upper or lower critical solution temperature, thus detaching cell sheet layer 20, and rinsed with an isotonic solution to collect cell sheet layer 20.

In another embodiment, cell sheet layer 20 is produced by culturing cells 22 on a non-adherent surface at sufficient densities. This provides a cell sheet layer 20 that has only a few structural defects as they are recovered with intracellular desmosome structures and the cell-to-cell connectivity and orientation being kept intact.

In this embodiment, cell growth support device 14 can be inherently non-adherent or can be rendered non-adherent by surface coatings well known to those skilled in the art. Commercially available cell growth support devices include, for example, the range of Corning® Ultra Low Attachment surface cell culturing products (Corning Inc., Corning N.Y.). These products have a hydrogel layer that is hydrophilic and neutrally charged covalently bound to polystyrene surfaces. Since proteins and other biomolecules passively adsorb to polystyrene surfaces through either hydrophobic or ionic interactions, this hydrogel surface naturally inhibits nonspecific immobilization via these forces, thus inhibiting subsequent cell attachment. Other biocompatible non-adherent materials include ePTFE, polystyrene, stainless steel, and some cross-linked cellulose derivatives. Examples thereof include cross-linked hydroxyalkyl celluloses e.g. hydroxyethyl cellulose, hydroxypropyl cellulose, methyl, thyl and methyl thyl celluloses. Cross-linked carboxyalkyl celluloses also included are carboxymethyl cellulose cross-linked with ethylene glycol diglycidyl ether (EGDGE) or 1,4 butanediol diglycidyl ether. Other materials include polyvinyl alcohol, poly(2-hydroxyethyl methacrylate) (Cellform® (MP Biomedicals, Irvine, Calif.), agarose, and crosslinked agarose.

To produce cell sheet layer 20, a cell population 22 (homogenous or heterogeneous) is cultured on a non-adherent substrate in the presence of commonly available culture media components, to promote extracellular matrix protein production. After an extended culture period, enough extracellular matrix protein is produced to make a coherent cell sheet.

Cells can be seeded on cell growth support device 14 at different densities sufficient to permit the formation of cell sheet layer 20. This will vary for different cell types and will need to be optimized. In the case of chondrocytes, the cell densities can range from 1,000 cells/cm² to 100,000 cells/cm². Appropriate culture medium (for example, D-MEM medium, MEM medium, HamF12 medium, HamF10 medium) is added to support device 14. Cells 22 of the required density are then added to support device 14 so that cells 22 settle to the bottom of the dish. Alternatively cells 22 of the required number can be suspended in the culture medium and added to support device 14. In this case, cells will not attach to the bottom of the dish, and cell-to-cell adhesion creates cell sheet layer 20. Cultures are maintained for a few days to a few weeks before cell sheet layer 20 can be recovered from support device 14. During culture, the culture medium may be exchanged, if needed. Usually, the culture medium is exchanged every 0.6 to 2 days of the culture. The addition of agents that promote cell growth, viability and/or cell-to-cell adhesion can be used during the culture process. For example, addition of agents such as ascorbic acid, retinoic acid, and copper can be used to increase the production of extracellular matrix proteins thereby generating a more robust sheet layer. Growth factors capable of stimulation of extracellular matrix protein production could be used or a combination of growth factors, microelements, vitamins and such.

Cell sheet layer 20 produced on the cell growth support device 14 can be recovered from support device 14 by gently peeling cell sheet layer 20 using a pair of forceps. Alternatively, cell sheet layer 20 is brought into close contact with a polymer membrane such that cell sheet layer 20 adheres to the polymer sheet. The coupled cell sheet/polymer backing can then be removed with a pair of tweezers. Peeling of cell sheet layer 20 can be performed not only in the culture solution used to culture cells 22, but also in other isotonic solutions. A suitable solution can be chosen in accordance with a specific object. Examples of the polymer membrane that can be used to achieve close contact with cell sheet layer 20 include polyvinylidene difluoride (PVDF), polypropylene, polyethylene, cellulose and its derivatives, as well as chitin, chitosan, collagen, polyurethane, and other such films or meshes made with known bioresorbable natural and synthetic polymers. The backing layer may be continuous or apertured (formed into a net). The backing layer may be flat or contoured. The contours may be produced for example by embossing. Suitably contoured films may also have apertures.

In another embodiment, cell sheet layer 20 can be comprised of reinforced cell sheets. Reinforced cell sheets can be formed by placing biodegradable or non-biodegradable reinforcing members at the bottom of support device 14 prior to seeding support device 14 with cells 22.

Reinforcing members include, but are not limited to, textile structures such as weaves, knits, braids, perforated films, meshes, and non-wovens. Cell sheet layer 20 that result will have incorporated the reinforcing member providing additional strength to cell sheet layer 20, which can be manipulated without the requirement for a backing layer. A preferred reinforcing member is a knitted or non-woven mesh comprised of poly(glycolic acid-co-lactic acid) copolymer, or PGA/PLA, fibers sold under the tradename VICRYL (Ethicon, Inc., Somerville, N.J.). The mesh can be placed at the bottom of the Corning® Ultra low attachment dish. Cells can then be seeded on to the dishes such that they will form cell-cell interactions but also bind to the mesh when they interact with the mesh. This will give rise to reinforced cell sheets with better strength and handling characteristics. Such reinforced cell sheets will not require additional backing polymer or scaffold to aid in removal.

Cell sheet layer 20 can also be made with a ring of the mesh such that the center of the cell sheet has no mesh and only the surrounding is reinforced with the mesh.

Cell sheet layer 20 is disposed on a scaffold layer 30 in the invention. Scaffold layer 30 is a three-dimensional scaffold, or framework. Scaffold layer 30 may be configured into various shapes such as generally flat, generally cylindrical or tubular, or can be completely free-form as may be required or desired for the corrective structure under consideration. When grown in this three-dimensional system, the proliferating cells from cell sheet layer 20 mature and segregate properly to form components of adult tissues analogous to counterparts found naturally in vivo.

For example, but not by way of limitation, scaffold layer 30 may be designed such that the scaffold structure: (1) supports the cell sheet without subsequent degradation; (2) supports cell sheet from the time of seeding until the tissue transplant is remodeled by the host tissue; (3) allows the seeded cell sheet to attach, proliferate, and develop into a tissue structure having sufficient mechanical integrity to support itself in vitro, at which point, the matrix is degraded.

Examples of scaffolds that may be used for aspects of the invention include textile structures such as weaves, knits, braids, meshes, non-wovens, and warped knits; porous foams, semi-porous foams, and perforated films or sheets and composite structures being a combination of the above structures. Non-woven mats may, for example, be formed using fibers comprised of natural or synthetic polymers.

Scaffold layer 30 of the present invention is preferably formed from a biocompatible polymer. A variety of biocompatible polymers can be used to form the mats and foams according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring.

Biocompatible polymers for use in the present invention may be biodegradable or non-biodegradable. Biodegradable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

In embodiments where the scaffold includes at least one synthetic polymer, suitable biocompatible synthetic polymers can include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof. Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of monomers including lactide (which includes lactic acid, D-, L- and meso lactide); glycolide (including glycolic acid); epsilon-caprolactone; p-dioxanone(1,4-dioxan-2-one); trimethylene carbonate(1,3-dioxan-2-one); alkyl derivatives of trimethylene carbonate; and blends thereof. Aliphatic polyesters used in the present invention can be homopolymers or copolymers (random, block, segmented, tapered blocks, graft, triblock, etc.) having a linear, branched or star structure.

In embodiments where the scaffold includes at least one natural polymer, suitable examples of natural polymers include, but are not limited to, fibrin-based materials, collagen-based materials, hyaluronic acid-based materials, glycoprotein-based materials, cellulose-based materials, silks and combinations thereof. By way of non-limiting example, the biocompatible scaffold can include a collagen-based small intestine submucosa, periosteal membrane, synovial or amniotic membrane.

One skilled in the art will appreciate that the selection of a suitable material for forming the biocompatible scaffold layer 30 of the present invention depends on several factors. These factors include in vivo mechanical performance; cell response to the material in terms of cell attachment, proliferation, migration and differentiation; biocompatibility; and optionally, biodegradation kinetics. Other relevant factors include the chemical composition, spatial distribution of the constituents, the molecular weight of the polymer, and the degree of crystallinity.

In one embodiment of the present invention, foams, composed of, for example, poly(epsilon-caprolactone-co-glycolic acid) copolymer, formed by processes such as freeze-drying, or lyophilization, as discussed in U.S. Pat. No. 6,355,699, can serve as scaffold 30. In another preferred embodiment cell sheets 20 of the invention are seeded onto foam scaffolds 30 that may be composite structures of fiber reinforced foams.

In another embodiment, textiles including weaves, non-wovens, knits, warped knits (i.e., lace-like structures), and braids are used as scaffold 30. In an exemplary embodiment the reinforcing component has a mesh-like structure. The fibers used to make the textile structures can be monofilaments, yarns, threads, braids, or bundles of fibers. These fibers can be made of any biocompatible material including synthetic biodegradable materials such as polylactic acid (PLA), polyglycolic acid (PGA), polycaprolactone (PCL), polydioxanone (PDO), polytrimethylene carbonate (PTMC), polyvinyl alcohol (PVA), copolymers or blends thereof In one embodiment, the fibers are formed of a polylactic acid and polyglycolic acid copolymer at a 95:5 mole ratio. The fibers can alternatively be made of a bioabsorbable glass. Bioglass, a silicate containing calcium phosphate glass, or calcium phosphate glass with varying amounts of iron particles added to control resorption time are examples of materials that could be spun into glass fibers and used for the textile structures. Also, filaments that form the fibers may be co-extruded to produce a filament with a sheath/core construction. Such filaments are comprised of a sheath of biodegradable polymer that surrounds one or more cores comprised of another biodegradable polymer. Filaments with a fast-absorbing sheath surrounding a slower-absorbing core may be desirable in instances where extended support is necessary for tissue ingrowth. Bioresorbable metals could be used as well.

In yet another embodiment, scaffold 30 is a felt, which can be composed of a multifilament yarn made from a bioabsorbable material, e.g., PGA, PLA, PCL copolymers or blends, or hyaluronic acid. One yarn of interest is made of poly(glycolic acid-co-lactic acid) copolymer or PLGA, sold under the tradename VICRYL (Ethicon, Inc., Somerville, N.J.) are used to form a mat. The yarn is made into a felt using standard textile processing techniques consisting of crimping, cutting, carding and needling.

In still yet another embodiment, scaffold 30 may be formed from thin, perforation-containing polymer sheets with perforations to allow tissue ingrowth. Such sheets could be made of the same polymers and copolymers as described above for scaffold materials.

One of ordinary skill in the art will appreciate that one or more layers of the scaffold 30 may be used in tissue engineering device 10 of the invention. In addition, scaffold layers 30 (e.g., meshes) of the same structure and chemistry or different structures and chemistries can be overlaid on top of one another to fabricate tissue engineering devices 10 with superior mechanical strength. The scaffold 30 might provide additional benefit since it might incorporate a genetic material, cytokines, and growth factors to promote survival, proliferation and differentiation of cells 22. Various means of incorporation of these beneficial factors into the scaffold 30 are known to those skilled in the art including but not limited to coating, Also, cells sheet layers 20 composed of different selected cells 22 can be overlaid on top of one another to fabricate tissue engineering devices 10 with superior cellular performance. Multilayered cell sheets can be made in several ways. For example, a first cell sheet layer of a first cell type is laid down on a non-adherent surface; then, a second cell sheet layer of a second cell type is laid down on the first cell sheet layer and allowed to adhere to the first cell sheet layer. The kinds of cells of the cell sheet layers to be laminated may be the same or different. For example, a cellular sheet or multi-layered sheet could be made of bladder derived urothelial cells and positioned on a luminar surface of the scaffold and smooth muscle cell sheet positioned on an external surface of the scaffold. In addition, keratinocytes may be overlaid with a fibroblast sheet and/or angioendothelial cell sheet that have been prepared by similar operations. This technique is extremely effective in realizing a product even nearer to an in vivo skin tissue. Sprinkling the sheet with endothelial cells, for example, helps to grow vascular networks (vasovasorum).

The number of cell sheet layers to be laminated is not limited. Generally the number cell sheet layers to be laminated is from 1 to 10, preferably from 1 to 5, more preferably from 1 to 3.

Scaffold 30 may be molded into a useful shape, such as a specific structure in the body to be repaired, replaced, or augmented.

The tissue engineering device 10 contemplated for use herein can be implanted in combination with any one of growth factors, drugs, or other components, such as bioactive agents that promote healing, or in growth of tissue, or stimulate vascularization or innervation or immunomodulatory agent thereof or otherwise enhance or improve the therapeutic outcome or the practice of the invention, in addition to cell sheets 20 of the invention.

In some embodiments, it is useful to re-create in culture the cellular microenvironment found in vivo, such that the extent to which the cells are grown prior to implantation in vivo or use in vitro may vary. Cell sheets 20 may be disposed onto scaffold 30 before or after forming the shape desired for implantation, e.g., ropes, tubes, filaments, and the like. Following disposition of cell sheet 20 onto scaffold 30, scaffold 30 is preferably incubated in an appropriate growth medium. During the incubation period, cells 22 in cell sheet 20 will grow and envelop scaffold 30 and may, for example, bridge, or partially bridge, any interstitial spaces therein. It is preferable, but not required to grow the cells to an appropriate degree that reflects the in vivo cell density of the tissue being repaired or regenerated.

In another aspect, cell sheet layer 20 of the invention is substantially decellularized to provide extracellular matrix materials provided by the population of cells. In some cases, it may be advantageous to decellularize or denature all or part of the tissue engineered device. A decellularized sheet may have a reduced level of immunogenicity, and may provide a better matrix. Decellularizing or denaturing tissue engineering device 10 may also enhance the mechanical characteristics of the device. Cell sheet layer 20 may be decellularized, denatured, or chemically modified using a variety of techniques. In the simplest embodiment, tissue engineering device 10 can be air-dried or lyophilized to kill the cells. Thermal shock, acoustic treatment, changes in pH, osmotic shock, mechanical disruption, or addition of toxins can also induce cell death or apoptosis. Similarly, tissue engineering device 10 can be cross-linked or fixed using agents such as paraformaldehyde. Other treatments to decellularize or denature the tissue are possible using radiation, detergents (SDS or triton x100), enzymes (RNAase, DNAase), or solvents (alcohol, acetone, or chloroform). Treatment with hypotonic and/or hypertonic solutions, which have nonphysiological ionic strengths, can promote the decellularization process. These various decellularization solutions generally are suitable as treatment solutions. Proteases also can be used effectively to decellularize tissue. These techniques are only some of the examples of techniques to decellularize, denature or chemically modify all or part of the tissue and are not meant to limit the scope of the invention.

The decellularization can be performed in stages with some or all of the stages involving differential treatments. For example, a potent mixture of proteases, nucleases and phospholipases could be used in high concentrations to decellularize the tissue. The decellularized extracellular matrix may then have applied another tissue sheet or another decellularized sheet. For example, one can roll a living layer on top of a decellularized layer.

Tissue engineering device 10 produced using this novel superior method can be an integral part of the new field of tissue engineering. Device 10 may be used to bioengineer tissue substitutes for other target tissues and organs in the body, including but not limited to, spinal cord, nerves, bone, cartilage, ligaments, tendons, heart valves, esophagus, cervix, bladder, lung tissue, kidney, skin, fascia, and other tissues. Create skin, blood vessels, myocardial patches, vascular patches, heart valves, and more complex organs. The tissue engineering device 10 could function on its own or could be a component of another system. For example based on a cell type it could provide an element of an extracorporeal devices simulating a specific organ function. For example, an extracorporeal kidney filtration unit. This tissue engineering device could function as a reservoir of functional beneficial proteins i.e. EPO.

Tissue engineering device 10 of the present invention could be supplied crypreserved or freeze-dried by using excipients known in the art such as trehalose, glycerol, sorbitol.

Tissue engineering device 10 of the present invention allows for the first time the production of mechanically sound living tissues and organs, that can be made exclusively from cell sheets. Furthermore, sheet-based tissue engineering opens the door to the production or tissues and organs made from patients own cells, avoiding all rejection complications.

The three-dimensional tissue structure can be also be used for, for example, in vitro permeability tests of drugs, and can also be utilized as a substitute model for an animal experiment or as an organ for transplantation.

### EXAMPLE 1

Cells were subjected to cell sheet preparation and were combined with non-woven, degradable scaffolds as a first step towards generation of tissue engineering devicces. Osteoblasts/chondrocytes for musculo-skeletal applications as well as urothelial cells/bladder smooth muscle cells for uro-genital applications were tested. Cells were cultured prior to scaffold deposition.

Porcine urothelial cells were isolated as described in Rahman, Z et al., “Isolation and primary culture urothelial cells from normal human bladder,” Urol. Res. 15 (1987) (6), pp. 315-320. Cells were cultured in Keratinocyte Serum Free Medium (Invitrogen, Carlsbad, Calif.) in tissue culture plastic flasks. The cultures were incubated at 37° C. with 5% CO₂. Cells used for experiments were between passages 1 and 2.

Human bladder smooth muscle cells were purchased from Cambrex (CC-2533; Cambrex, Walkersville, Md.). Cells were cultured in muscle cell growth medium, SmGM-2 (CC-3182; Cambrex, Walkersville, Md.) in tissue culture plastic flasks. The cultures were incubated at 37° C. with 5% CO₂. Cells used for experiments were between passages 3 and 5.

Human osteoblast cells were purchased from Cambrex (CC-2533, Cambrex, Walkersville, Md.). Cells were cultured in smooth osteoblast growth medium, OGM (CC-3207; Cambrex, Walkersville, Md.) in tissue culture plastic flasks. The cultures were incubated at 37° C. with 5% CO₂. Cells used for experiments were between passages 3 and 4.

Bovine cartilage tissue was harvested from femoral condyles of adult bovine animals. Primary bovine chondrocytes were isolated from cartilage tissue by O/N collagenase digestion at 4° C. Cells were cultured in chondrocyte growth medium, CGM (CC-3216; Cambrex, Walkersville, Md.) in tissue culture plastic flasks. The cultures were incubated at 37° C. with 5% CO₂. Cells used for experiments were between passages 3 and 4.

The cell types described above were also cultured in sheets on thermoresponsive dishes. Cells were seeded on 35 millimeter p(NIPAAM)-coated dishes supplied by CellSeed, Inc. (Tokyo, Japan). CellSeed, Inc. manufactures several types of dishes from A to G varying in the degree of cellular attachment or detachment. Dishes range from good cellular attachment to good cellular detachment to strong cellular detachment. Cells were grown to confluency as shown in Table 1 below.

TABLE 1
Time to confluence and thermoresponsive dish type are listed for the
appropriate cell type.

| Cell type | Dish type | Time to confluence |
|---|---|---|
| Chondrocytes | C at 10,000 | 6 days |
| | cells/cm² | |
| Osteoblasts | D at 10,000 | 7 days |
| | cells/cm² | |
| Urothelial cells | D at 10,000 | 8 days |
| | cells/cm² | |
| SMCs | B at 10,000 | 5 days |
| | cells/cm² | |

The scaffold material was the same for all of the treatment groups and consists of a PGA non-woven mesh at 55 grams/cubic centimeter and thickness of 2 millimeters. Fiber size is 12-15 microns. Scaffolds were coated with a 5% solution of 50/50 mol percent PLGA (Sigma, St. Louis, Mo., Mw 40,000-75,000) in methylene chloride (Sigma Aldrich, St. Louis, Mo.), which made them stiff. Scaffolds were cut in the form of 35 millimeter diameter circles and were sterilized using ethylene oxide sterilization. Next, the cell sheets were co-cultured with the scaffold. In the case of single layer cell sheets, the cell sheet for the first cell type was embedded by adding 500 microliters of fibrinogen (80 milligrams/milliliter in phosphate buffered saline (PBS) (Sigma, St. Louis, Mo.) and gelatinized with 400 microliters of bovine thrombin solution (Jones Pharma, Inc., Middleton, Wis.) while still in the thermoresponsive dish. The embedded cell sheet was then layered on top of the scaffold in a 100 millimeter tissue culture polystyrene dish. 30 milliliters of growth medium were added to the dish and the dish was placed in an incubator for two days. The appropriate medium was used for the given cell type as shown in Table 2 below. The medium was changed twice daily. After incubation, the device was turned over and the process was repeated on the other side of the scaffold with the second cell type. 30 milliliters of growth medium were added to the dish and the dish was placed in an incubator for four days. Therefore, the total incubation time for the scaffold devices was 6 days.

TABLE 2
The appropriate cell growth medium for cell types during
scaffold deposition.

| Cell Type | Growth Medium |
|---|---|
| Chondrocytes | Osteoblasts growth medium (cat#: CC-3207, Cambrex, |
| alone | Walkersville, MD) supplemented with non-essential |
| | amino acids (NEAA, cat#: 11140-050, Invitrogen, |
| | Carlsbad, CA) at a concentration of 0.1 millimolar and |
| | HEPES buffer (cat#: 15630-080, Invitrogen, Carlsbad, |
| | CA) at a concentration of 10 millimolar |
| Chondrocytes and | Osteoblasts growth medium (cat#: CC-3207, Cambrex, |
| osteoblasts | Walkersville, MD) |
| Bladder smooth | Dulbeco Modified Eagle's Medium (DMEM), 10% |
| muscle cells alone | fetal bovine serum, penicillin/streptomycin (Invitrogen, |
| | Carlsbad, CA) |
| Bladder smooth | 50:50 mixture of Keratinocyte Serum Free Medium |
| muscle cells with | and DMEM, 10% FBS, penicillin/streptomycin |
| urothelial cells | (Invitrogen, Carlsbad, CA) |

In the case of multi-layer cell sheets, the first cell sheet was embedded and gelatinized as described above for single sheets. The cell sheet was transferred on top of a second cell sheet in a thermoresponsive dish and the two sheets were lifted out of the dish and transferred on top of another sheet. The cell sheets were layered in this manner until the desired number of sheets was obtained. Once the desired number of sheets was obtained the multi-layer sheet was incubated for approximately 45 minutes then transferred onto a scaffold in a 100 millimeter tissue culture polystyrene dish, embedded with 500 microliters of fibrinogen (80 milligrams/milliliter in PBS) and gelatinized with 400 microliters of thrombin solution. 30 milliliters of growth medium were added to the dish and the dish was placed in an incubator for two days. The appropriate medium was used for the given cell type as shown in Table 2. The medium was changed twice daily. After incubation, the device was turned over and the process was repeated on the other side of the scaffold with the second cell type. 30 milliliters of growth medium were added to the dish and the dish was placed in an incubator for four days. The total incubation time for the scaffold devices was 6 days.

Cell suspensions were seeded on the scaffold such that the cell number was matched to corresponding cell sheets as shown in Table 3. The cells were resuspended in 1 milliliter of medium. The 1 milliliter of cell suspension was placed in the middle of the scaffold and incubated for 45 minutes to allow cell attachment to the scaffolds. The appropriate medium was used for the given cell type as shown in Table 2. The medium was changed twice daily. After incubation, the device was turned over and the process was repeated on the other side of the scaffold with the second cell type. Then, 30 milliliters of medium was added to the dish and placed in an incubator for four days. The total incubation time for the scaffold devices was 6 days.

TABLE 3
Seeding density for cell sheets as well as viability of cells
seeded onto the scaffolds.

| Cell type | Number of cells seeded | Viability |
|---|---|---|
| Chondrocytes | 1.53 × 10⁶ (two sheets) | 85% |
| Osteoblasts | 6.45 × 10⁵ (two sheets) | 80% |
| Urothelial cells | 5.03 × 10⁵ (one sheet) | 84% |
| | 1.51 × 10⁶ (3 sheets) | |
| SMCs | 4.35 × 10⁵ (one sheet) | 92% |
| | 1.30 × 10⁶ (3 sheets) | |

The following cell/scaffold devices were generated using the methods described above:

1. One Urothelial cell sheet+Scaffold+one Bovine Smooth Muscle (BSM) cell sheet

2. One cell sheet equivalent of Urothelial cell suspension+Scaffold+one cell sheet equivalent of BSM cell suspension

3. Two Osteoblasts cell sheets+Scaffold+two Chondrocytes cell sheets

4. Two cell sheet equivalents of Osteoblasts cell suspension+Scaffold+two cell sheet equivalents of Chondrocytes cell suspension

5. Three Urothelial cell sheets+Scaffold+three Bovine Smooth Muscle (BSM) cell sheets

6. Three cell sheet equivalents of Urothelial cells suspension+Scaffold+three cell sheet equivalents of BSM cell suspension

7. Scaffold alone

Three days after the last cell deposition, the devices were cut into approximately 0.5 cm² pieces using a scalpel. The pieces were embedded in fibrin glue by adding 30 microliters of fibrinogen (80 milligrams/milliliter in PBS) and gelatinized with 5 microliters of bovine thrombin solution (Jones Pharma, Inc., Middleton, Wis.). The embedded and gelatinized device pieces were stored at 37° C. in the culture medium overnight. The device pieces were removed from the culture medium and washed with PBS and characterized by live/dead staining and histology.

Live/dead staining allows visualization of cells remaining in the scaffolds after all manipulations as well as to examine the degree of cell survival within the scaffolds. Cells were exposed to 1 micromolar Calcein AM (Molecular Probes, Eugene Oreg.) in PBS (live staining) and 1 micromolar Ethidium (Molecular Probes, Eugene Oreg.) in PBS (dead staining) concurrently for 1 hour at room temperature. Following staining, fluorescence was visualized using the appropriate fluorescence filter on an Olympus inverted epi-fluorescent microscope (Olympus, Melville, N.Y.). Representative images were captured using a digital color videocamera and ImagePro software (Media Cybernetics, Carlsbad, Calif.). The majority of stained cells were viable with only a few dead cells. Furthermore, the live cells were dispersed throughout the scaffold without apparent difference between the two seeding methods.

Histology samples were fixed in 10% formalin and were sent to Paragon Bioservices (Baltimore, Md.) for paraffin embedding, sectioning, and staining with Hematoxylin/Eosin (H/E) and Safranin O (SO). H/E staining also confirmed that cells were present throughout the scaffolds regardless of seeding method. Furthermore, for scaffolds seeded with one cell sheet versus three cell sheets (urothelial cells/BMS cells) there was no apparent difference in cell distribution. Safranin O staining revealed similar results. No Safranin O positive areas were detected in any of the examined scaffolds. This suggests that in vitro culture of cell/scaffold devices was not sufficient for generation of detectable levels of proteoglycans, regardless of seeding method.

Cells were subjected to cell sheet preparation and were combined with non-woven, degradable scaffolds as a first step towards generation of tissue engineering devices. Two cell systems were tested: osteoblasts/chondrocytes for potential regenerative medicine musculo-skeletal systems as well as urothelial cells/bladder smooth muscle cells for uro-genital applications. The seeding of cells on scaffolds using cell sheets was compared against the conventional method of seeding scaffolds with cell suspension. Seeding of a biodegradable, biocompatible scaffold with cell sheets provides equivalent cell viability and cell distribution to seeding by cell suspension. Therefore, seeding scaffolds using the cell sheet method is a good alternative to the conventional method of cell suspension seeding with the benefit of one-step application and more controlled device handling.

### EXAMPLE 2

An in vivo implantation pilot study was performed using the tissue engineering devices prepared in Example 1 in a SCID mouse subcutaneous model. Twenty Fox Chase SCID CB17SC/male mice were ordered from Taconic Inc. (Germantown, N.J.). The average age of the animals at the time of the study was 5 weeks. Animals were selected without any apparent systematic bias. Duration of the study was 4 weeks.

The devices were implanted subcutaneously by making two skin incisions, each approximately 5 mm in length, on the dorsum of the mice. The incisions were placed transversely over the lumbar area about 5 mm cranial to the palpated iliac crest, with one on either side of the midline. Devices with the same treatment were placed in the two sites. The skin was separated from the underlying connective tissue to make a small pocket, and the implant was placed about 10 mm cranial to the incision. The skin incisions were closed with Reflex 7 metal wound clips (CellPoint Scientific, Inc., Gaithersburg, Md.).

All mice survived the surgery and the duration of the study. Mice were euthanized by CO₂ inhalation at 4 weeks post-implantation. Gross observations of the implanted sites were recorded. The subcutaneous implantation sites with their overlying skin were excised. With the exception of scaffolds seeded with osteoblasts/chondrocytes, subcutaneous implantation sites with their overlying skin were bisected. Half of the tissue was preserved in 10% buffered formalin fixative for paraffin embedding and histological staining (H&E). Another half was frozen for immunohistochemical staining (pancytokeratin, keratin 7, anti-alpha smooth muscle actin). The samples will be evaluated histologically for retention of cell phenotypes, cellular infiltration into the scaffold and vascularization. Scaffolds seeded with osteoblasts/chondrocytes were preserved in 10% buffered formalin fixative for paraffin embedding and histological staining only (H&E and Safranin O).

The gross observations were noted as follows: upon sacrifice at 4 weeks post-implantation the implants appeared firm to the touch, which indicated tissue ingrowth. The implants maintained their shape and in some cases were partially resorbed. Implants also appeared pinkish in color, which indicated vascularization.

Although this invention has been shown and described with respect to detailed embodiments thereof, it will be understood by those skilled in the art that various changes in form and detail thereof may be made without departing from the spirit and scope of the claimed invention.

What is claimed is:

1. A tissue engineering device for use in the repair or regeneration of tissue comprised of a support scaffold layer and a cell sheet layer.

2. The tissue engineering device of claim 1 wherein said cell sheet layer is comprised of cells selected from the group consisting of chondrocytes, osteoblasts, fibroblasts, angioblasts, myoblasts, epithelial cells, urothelial cells, smooth muscle cells, keratinocytes, beta cells, endothelial cells, fibrocytes, vascular endothelial cells, hepatocytes, small intestine epithelial cells, epidermal keratinocytes, bone marrow mesenchymal cells, cardiomyocytes, intervertebral disc cells, oral mucosal epithelia, gastrointestinal mucosal epithelia, urinary tract epithelia, skeletal joint synovium, periosteum, perichondrium, skeletal muscle cells, smooth muscle cells, cardiac muscle cells, pericardium, dura, meninges, keratinocyte precursor cells, pericytes, glial cells, neural cells, amniotic membrane, placental membrane, serosal cells, undifferentiated stem cells, undifferentiated progenitor cells, pre-differentiated stem cells, pre-differentiated progenitor cells, neural stem cells, neural progenitor cells, neuronal cells, dendritic cells, and genetically engineered cells.

3. The tissue engineering device of claim 1 wherein said cell sheet layer is comprised of stem cells.

4. The tissue engineering device of claim 3 wherein said stem cells are selected from the group consisting of hematopoietic, mesenchymal, postpartum, pancreatic, hepatic, retinal epithelial, olfactory bulb, endothelial, muscle, adipose-derived, ileac crest, bone marrow, periodontal ligament, oval and dermal cells and organ specific stem and progenitor cells.

5. The tissue engineering device of claim 1 wherein said cell sheet layer further comprises extracellular matrix protein.

6. The tissue engineering device of claim 5 wherein said extracellular matrix protein is selected from the group consisting of collagens, laminins, fibronectin, vitronectin, tenascin, integrins, hyaluronic acid, chondroitin sulfate, dermatan sulfate, heparan sulfate, heparin, keratan sulfate, chitosan, cellulose, oxidized regenerated cellulose, elastin and fibrin.

7. The tissue engineering device of claim 1 wherein said cell sheet layer further comprises growth factor.

8. The tissue engineering device of claim 7 wherein said growth factor is selected from the group consisting of vascular endothelial cell growth factor, platelet derived growth factor, epidermal growth factor, fibroblast growth factor, hepatocyte growth factor, insulin-like growth factor, brain derived neurotrophic factor, growth differentiation factor-5, EPO, and transforming growth factor.

9. The tissue engineering device of claim 1 wherein said cell sheet layer further comprises a reinforcing member.

10. The tissue engineering device of claim 9 wherein said reinforcing member is selected from the group consisting of weave, knit, braid, film, perforated film, mesh, and non-woven.

11. The tissue engineering device of claim 9 wherein said reinforcing member is a non-woven mesh comprised of poly(glycolic acid)/poly(lactic acid) copolymer fibers.

12. The tissue engineering device of claim 1 wherein said support scaffold layer comprises a biodegradable homopolymer, copolymer or polymer blend comprised of monomers selected from the group consisting of lactide, glycolide, epsilon-caprolactone, 1,4-dioxan-2-one, 1,3-dioxan-2-one, and alkyl derivatives of trimethylene carbonate.

13. The tissue engineering device of claim 1 wherein said support scaffold layer comprises a polymer selected from the group consisting of fibrin-based material, collagen-based material, hyaluronic acid-based material, glycoprotein-based material, cellulose-based material, and silk.

14. The tissue engineering device of claim 1 wherein said support scaffold layer is selected from the group consisting of weaves, knits, braids, non-wovens, warped knits, meshes, porous foams, semiporous foams, and perforated sheets.

15. The tissue engineering device of claim 1 wherein said support scaffold layer is a porous foams comprised of poly(epsilon-caprolactone)/poly(glycolic acid) copolymer.

16. The tissue engineering device of claim 1 wherein said support scaffold layer is a non-woven mesh comprised of polyglycolic acid.

17. The tissue engineering device of claim 16 wherein said support scaffold layer is coated with 50/50 mol percent poly(lactic acid-co-glycolic acid).
